(19) United States
(12) Patent Application Publication
(10) Pub. No.: US 2007/0196463 A1
(43) Pub. Date: Aug. 23, 2007

(54) Enteric Soft Gelatin Capsule Containing Esomerpazole And Method Of Preparation

(75) Inventors: Khadgapathi Podili, Hyderabad (IN); Venkaiah Chowdary Nannapaneni, Hyderabad (IN)

Correspondence Address: MERCHANT & GOULD PC, P.O. BOX 2903, MINNEAPOLIS, MN, 55402-0903, US

(73) Assignee: Natco Pharma Limited, Hyderabad (IN)

(21) Appl. No.: 10/572,984

(22) PCT Filed: Sep. 25, 2004
(86) PCT No.: PCT/IN03/00323

§ 371 Date: Nov. 7, 2006

(51) Int. Cl. A61K 31/4439; A61K 9/64

(52) U.S. Cl. 424/456; 514/338

(57) ABSTRACT

The present invention relates to an improved pharmaceutical composition, in the form of a soft gel capsule resistant to digestive juice. The composition of the present invention is made up of gelatin and an enteric polymer in the form of free acid or its salt, containing used in the treatment of duodenal ulcers, solublised and/or suspended in a liquid or semisolid medium, comprising the enantiomers of omeprazole such as esomeprazole or rabeprazole or other enantiomers of omeprazole or its salts or derivatives or their mixtures, a hydrophobic carrier, an alkaline inert reacting material and a surface active agent and/or a solublising agent wherein the resulting capsules being insoluble in aqueous medium up to a pH of 5.5 but quickly dissolving above pH of 6.0. The present invention also relates to a process of preparing the above said pharmaceutical composition.

The present invention relates to an improved pharmaceutical composition and a process for its preparation. The present invention particularly relates to an improved pharmaceutical composition, in the form of a soft gel capsule resistant to digestive juice. The composition of the present invention is made up of gelatin and an enteric polymer in the form of free acid or its salt, containing a benzimidazole derivative used in the treatment of duodenal ulcers, solublised and/or suspended in a liquid or semisolid medium, comprising of a hydrophobic carrier, an alkaline inert reacting material and a surface active agent and/or a solublising agent. The present invention more particularly relates to an improved pharmaceutical composition, in the form of a soft gel capsule resistant to digestive juice, also containing enantiomers of omeprazole such as esomeprazole or rabeprazole or other enantiomers of omeprazole or its salts or derivatives or their mixtures relates to a method for preparing the above said pharmaceutical composition. The invention also relates to a process for the preparation of the said composition.

Benzimidazole derivatives such as omeprazole, lansoprazole, timoprazole and pantoprazole etc., are known potent proton pump inhibitors with powerful inhibitory action against the secretion of gastric juice (Lancet, Nov. 27, 1982 pages 1223-1224). They are used in the treatment of Zollinger-Ellison syndrome and stress related esophagitis ulceration. The derivatives are well known and are described, for example in EP-A 0005129.

It has been found that these benzimidazole derivatives, and in particular omeprazole, esomeprazole, rabeprazole and the like, are susceptible to degradation in acid and neutral media. It is known to protect oral dosage forms of such benzimidazole derivatives by providing an enteric coating. In this way, the active material is protected from acidic gastric juices until it reaches the desired site of release, e.g. the small intestine. Because certain enteric coatings themselves can be, or contain, acidic material, it also often is required to protect the benzimidazole derivatives from the acidity of the enteric coating. For example, it is known to formulate the benzimidazole derivatives with an alkaline material before applying the enteric coating. It is also known to provide an intermediate coating between the benzimidazole derivative and the enteric coating. Generally the intermediate coating is selected so as to be substantially water-soluble or water-dispersible.

EP-A-024 7983; U.S. Pat. No. 4,786,505; U.S. Pat. No. 4,853,230 and U.S. Pat. No. 5,385,739 describe oral pharmaceutical preparations containing benzimidazole derivatives that are potent inhibitors of gastric acid secretion, which are composed of a core material in the form of small beads or tablets containing one of the benzimidazole derivatives, particularly omeprazole, together with an alkaline reacting compound. The core material contains one or more inert reacting sub-coating layers thereby providing a final outer enteric coating. Although the above-described compositions are reasonably stable over an extended period of storage, discoloration of the pellets and/or tablets with reduced gastric resistance and reduction of dissolution rate in alkaline buffers was observed.

Moreover the processes disclosed above are time-consuming and laborious, involving many stages in manufacturing of the composition, consequently increasing the cost of the final composition.

In a German patent DE 3,222,476 it has been described in which a soft gelatin capsule that is resistant to gastric juice, whose wall includes a usual gelatin mass which contains polyvinyl acetate phthalate, hydroxypropyl methyl cellulose phthalate or a vinyl acetate/crotonic acid copolymer and/or an alkali metal salt, ammonia salt or amino salt of the same in their wall, and which released its contents readily in the intestines within the prescribed time. The capsules are further treated on the surface with an aldehyde-coating agent.

Various compounds used in inhibiting gastric acid secretion are known in the art as mentioned above which include a class of benzimidazole-substituted compounds, one of which is omeprazole. Omeprazole is currently-commercially available in the formulation PRILOSEC.

In particular, U.S. Pat. No. 4,255,431 proposes such benzimidazole-substituted compounds particularly Omeprazole, and various methods of making these compounds are also proposed in '431 patent.

The active substance(s), benzimidazole derivatives, needs to be protected by a sub coat from the reacting acidic groups present in the enteric polymers. The processing time and the number of steps involved are many. The resulting product, i.e., pellets/beads/tablets, has to be dried to keep moisture content below 1.5% to ensure drug stability during processing and through its shelf storage. The active substance(s), benzimidazole derivatives, present in the final formulation as solid dispersed in a hydrophilic solid matrix and hence requires some time to dissolve into the surrounding intestinal fluid before being absorbed. Large quantities of polymer i.e. 15-25% w/w, based on product, need to be applied to achieve desired gastric protection. The pH of medium used to suspend/solublise the drug needs to be adjusted to alkaline condition i.e. above pH 8.0 to prevent degradation during processing. The micro environment surrounding the core also contains alkaline material to neutralise the acidic medium that permeates the outer enteric coating during the product transit through stomach. In case of pellets/beads large surface area needs to be coated with protective polymer sub-coat.

The U.S. Pat. No. 5,714,504 provides methods for the preparation of pure crystalline enantiomeric salts of omeprazole which is having a dosage strength of equivalent 20 mg base and equivalent 40 mg base in the form of oral delayed release tablets or granules.

The U.S. Pat. No. 5,877,192 describes a method for the treatment of gastric acid related diseases and production of medication using enantiomer of omeprazole by a method of inhibiting gastric acid secretion comprising the oral administration of a pharmaceutical compositions which is having a dosage strength of equivalent 20 mg base and equivalent 40 mg base in the form of oral delayed release tablets or granules.

Human testing of enantioners of omeprazole shows that the S-enantiomer is more active, according to studies conducted by Lindberg and others, the higher efficacy of esomeprazole is due to its higher and more consistent bioavailability compared with omeprazole. And because of the more consistent pharmacokinetics of esomeprazole, inter-individual variability with esomeprazole is reduced [Aliment. pharmacaol. Ther., 17,481(2003)].

The U.S. Pat. No. 6,328,993 provides a novel oral administration form as a proton pump inhibitor selected containing compounds selected from the a group consists of pantoprazole, omeprazole, esomeprazole, lansoprazole or rabeprazole as acid-labile active compounds in which the acid-labile active compound does not have to be protected by an enteric coating. As the above mentioned prior art shows, the preparation of oral administration forms for acid-labile active compounds of pantoprazole sodium sesquihydrate requires technically complicated process.

The U.S. Pat. No. 6,489,346 provides an oral solution/suspension comprising a proton pump inhibitor selected from omeprazole, lansoprazole, rabeprazole, esomeprazole, pantoprazole, pariprazole and lemiprazole or an enantiomer and at least one buffering agent. The liquid oral compositions can be further comprised of parietal cell activators, anti-foaming agents and/or flavoring agents.

The composition can alternatively be formulated as a powder, tablet, suspension tablet, chewable tablet, capsule, effervescent powder, effervescent tablet, pellets and granules.

In our co-pending Indian application no 968 MAS 99 and the corresponding PCT application no PCT/IN00/00079 we have disclosed a process for the production of soft gelatin capsules in a conventional manner using gelatin mass having an enteric polymer incorporated into it and to incorporate a mixture containing benzimidazole derivative, and an alkaline reacting substance with larger quantities of hydrophobic oily substance or a mixture of such oily substances into the gelatin shell. The resulting capsules being insoluble up to a pH value of 5.5 in aqueous media, but quickly dissolving above a pH of 6.0.

The said invention has been developed based on our finding, that the incorporation of benzimidazole derivatives, particularly useful for the treatment of duodenal ulcers, along with an alkaline inert reacting material into a hydrophobic oily substance wherein the benzimidazole derivative is in the form of solution or dispersion, results in extended periods of stability during which period the composition does not get discolored and/or degraded.

In other words, the active ingredient in the composition is kept partially in the form of solution and partially in the form of finely divided particles suspended freely in the oily substance which makes the active ingredient readily absorbable the moment the gastric resistant but intestinal soluble gelatin polymer composition is dissolved.

Such a composition will have an advantage over the existing form of the formulation as the available dosage forms for benzimidazole derivatives are having the total amount of active ingredient in the form of solid particles engulfed in a solid matrix of excipients preferably hydrophilic substances, further coated with protective and gastric resistant enteric polymer coatings.

The enantiomers of omeprazole such as esomeprazole, rabeprazole or its salts or its derivatives, its mixtures, especially esomeprazole, is/are used as the benzimidazole derivatives the resulting composition has also been found to have extended periods of stability during which period the composition does not get discolored and/or degraded.

None of the above said prior art discloses and/or envisages such a composition and therefore the composition of the present invention is unique and novel.

Accordingly the present invention provides, compositions containing the enantiomers of omeprazole such as esomeprazole, rabeprazole, its salts or its derivatives or its mixtures and a method of making the said composition that is not suggested by the prior art.

Considering the importance gained for the composition containing benzimidazole derivatives, particularly omeprazole, more particularly esomeprazole or rabeprazole and other enantiomers of omeprazole or its salts or derivatives or its mixtures, for the treatment of duodenal ulcers, there is a need for the development of pharmaceutical composition containing said derivatives having stability for an extended period during which period the composition does not get discoloured and/or degraded.

The present invention is directed to, the production of soft gelatin capsules in a conventional manner using gelatin mass in the known composition and to additionally incorporate substances into the gelatin shell which are insoluble up to a pH value of 5.5 in aqueous media, but quickly dissolve above a pH of 6.0.

According to the main objective of the present invention there is provided an intestine dissoluble soft gel capsule composition of enantiomers of omeprazole such as esomeprazole, rabeprazole or its salts or its derivatives or its mixtures.

According to another objective of the invention there is provided a pharmaceutical composition comprising the enantiomers of omeprazole such as esomeprazole, rabeprazole its salts, derivatives or its mixtures to be filled into soft gel capsules, which composition reduces degradation of the benzimidazole derivatives during storage/shelf life.

According to still another objective of the invention there is provided a process for preparation of soft gel capsules comprising enantiomers of omeprazole such as esomeprazole, rabeprazole its salts, derivatives or, its mixtures that are resistant to the digestive/gastric juice, a gelatin mass and an enteric polymer in the form of a free acid or as its salt.

Accordingly, the present invention provides, an improved pharmaceutical composition in the form of a soft gel capsule resistant to gastric juice and soluble in intestine useful for the treatment of duodenal ulcers and related ailments which comprises a gelatin shell which is resistant to gastric juice and soluble in intestine having an enteric polymer coating in the form of free acid or its salt, the capsule incorporating a composition comprising of enantiomers of omeprazole such as esomeprazole, rabeprazole its salts, derivatives or its mixtures a hydrophobic oily substance or a mixture of such oily substances, an alkaline inert reacting material, a dispersing agent, a surface active agent and/or a solublising agent; the resulting capsules being insoluble in aqueous medium up to a pH of 5.5 but quickly dissolving above pH of 6.0.

According to another feature of the present invention, there is provided a process for the preparation of a pharmaceutical composition in the form of a soft gel capsule resistant to gastric juice and soluble in intestine useful for the treatment of duodenal ulcers and related ailments which comprises forming a gelatin shell which is resistant to gastric juice and soluble in intestine having an enteric polymer coating in the form of free acid or its salt, incorporating into the resultant capsule a composition comprising of enantiomers of omeprazole such as esomeprazole, rabeprazole its salts, derivatives or its mixtures a hydrophobic oily substance or a mixture of such oily substances, an alkaline inert reacting material, a dispersing agent, a surface active agent and/or a solublising agent, the resulting capsules being insoluble in aqueous medium up to a pH of 5.5 but quickly dissolving above pH of 6.0.

The capsules so formed are insoluble in aqueous medium up to a pH of 5.5 but quickly dissolve above pH of 6.0.

In a preferred embodiment of the invention, the enteric polymer used in the soft gel capsule composition may be selected from among the polymers but not limited to free acid forms of hydroxypropyl methyl cellulose phthalate, alkylmethacrylate and methacrylic acid ester copolymers, polyvinylacetate phthalate and the like or their ammonia or alkali metal salts. The amount of such enteric polymer employed may range from 2.0-40.0 percent, preferably 5.0-25.0 percent by weight with reference to the dried shell.

The gelatin mass into which the enteric polymer is incorporated is made up of a composition known in the art and contains gelatin, a plasticizer, preservatives, colourants, opacifiers, flavours etc., as required.

In order to carry out faster dissolution of the enteric polymer for preparing the capsule shell composition, the polymer is first dispersed in water, then an aqueous solution of ammonia or alkali metal salt is mixed while stirring. When alkali metal salt is used it may be selected from substances such as sodium hydroxide, potassium hydroxide, bicarbonate sodium, potassium bicarbonate, sodium carbonate, potassium carbonate etc. The quantity of the base materials used is such that it is sufficient to neutralise 60 to 100 percent of the free acid groups present in the selected enteric polymer.

The excess ammonia or alkali has to be removed from the capsule shell composition to avoid decomposition of the ester couplings in enteric polymers. When aqueous ammonia solution is used to prepare polymer solution, the excess ammonia has to be removed before preparing the capsule after mixing with the gelatin mass by mixing the mass under reduced pressure in warm condition.

When alkali metal salts are used, the excess alkali is to be neutralized by treating the capsules with an acid selected from any of the following ones, hydrochloric acid, sulphuric acid, nitric acid, phosphoric acid, mono carboxylic acids such as acetic acid, propionic acid, benzoic acid etc., dicarboxylic acids such as oxalic acid, maleic acid, fumaric acid etc. The acids are used in the form of cold dilute aqueous solutions in the concentration range of 3 to 30% depending on the type of acid used. The acid treatment may be carried out after manufacturing and partial drying of the capsules to avoid deformation and/or leakage of the capsule contents.

According to another feature of the invention the soft gel capsules are optionally treated with a cross-linking agent that reacts with gelatin and makes it insoluble in gastric juice. The cross-linking agent may be selected from among the aldehydes such as formaldehyde, glutaraldehyde, crotonaldehyde 1,2-phthalic acid aldehyde, 1,3-phthalic acid aldehyde, 1,4-phthalic acid aldehyde or carbodimides like 1-ethyl-3-[2-morpholinyl-(4)-ethyl]-carbodiimide-metho-p-toluene-sulfonate. The treatment may be done by either coating 0.05 to 1.0% w/v of the substance in an alcohol containing aqueous solution on to the soft gel capsule surface or mixing these substances in the gelatin mass before capsule manufacturing.

According to another feature of the invention the pharmaceutical composition containing enantiomers of omeprazole such as esomeprazole, rabeprazole its salts, derivatives or its mixtures known for its potent proton pump inhibition with powerful inhibitory action against the secretion of gastric juice, is prepared by suspending and/or solubilising the enantiomers of omeprazole such as esomeprazole, rabeprazole its salts, derivatives or its mixtures in a carrier mixture composed of a hydrophobic oily carrier material, an alkaline inert reacting material and a dispersing agent and/or a surface active agent. The amount of esomeprazole or rabeprazole or other enantiomers of omeprazole or its salts or derivatives or their mixtures used is equivalent to one unit dose recommended depending on the esomeprazole or rabeprazole or other enantiomers of omeprazole or its salts or derivatives or their mixtures incorporated. The amount incorporated into enteric soft gel capsule may range from 5.0 to 100.0 mg per capsule, preferably 10.0 to 40.0 mg per capsule.

The hydrophobic oily material may be selected from among the following fats and oils: Fats and oils of vegetable origin such as sesame oil, corn, maize oil, soybean oil, sunflower oil, arachis oil, gingly oil etc.; animal oils such as fish oil, pig oil, beef oil etc.; esters of straight chained aliphatic oils contained in glycerol such as Sunsoft 700 P-2 (a monoester substance manufactured by Taiho Chemicals Company) Panasete 810 (a triester substance, manufactured by Nippon Oils and Fats); hydrogenated vegetable oils or a mixture thereof. The amount of such hydrophobic oily material may range from 25.0 to 95.0 percent, preferably 35.0 to 90 percent by weight with reference to the contents filled in a capsule.

The alkaline buffering material present in the pharmaceutical composition may be selected from among but are not restricted to substances such as the sodium, potassium, calcium, magnesium and aluminum salts of phosphoric acid, carbonic acid, citric acid, other suitable organic or inorganic acids; substances used in antacid preparations; meglumine, triethanolamine etc. The amount of such alkaline buffering material present in the composition may range from 2.0 to 40.0 percent, preferably 5.0 to 25.0 percent by weight with reference to the contents filled in capsule.

The substances that increase viscosity of the oily material either by dissolving or by forming a colloidal dispersion are used as dispersing agents. The dispersing agent is selected from among but not restricted to colloidal silicon dioxide, polyvinylpyrrolidone, microcrystalline cellulose etc. The amount of such suspending agent present in the composition may range from 0.5 to 20.0 percent preferably 1.0 to 10.0 percent by weight with reference to the content filled in capsules.

The surface active agent used as solublising and/or dispersing agents is selected from among but is not restricted to substances such as lecithin, polyoxyethylene castor oil derivative such as Cremophor RH 40 (polyoxyl 40 hydrogenated castor oil), Cremophor EL (polyoxyl 35 castor oil, BASF) polyoxyethylene sorbitan fatty acid esters, Gelucire 33/01 (glycerol esters of fatty acids), sodium lauryl sulphate, docusate sodium and the like. The amount of such surface active agent present in the composition may range from 2.0 to 20.0 percent preferably 4.0 to 15.0 percent by weight with reference to contents filled in capsule.

The seamless soft gel capsules can be manufactured on a rotary die machine filling with the liquid and/or semi solid composition containing esomeprazole or rabeprazole or other enantiomers of omeprazole or its salts or derivatives or their mixtures.

The invention is described in detail in the Examples given below which are provided by way of illustration only and therefore should not be construed to limit the scope of the invention.

### EXAMPLE—1

a) Composition of the Soft gelatin Shell:

| Name of the ingredient | Percent by wt. |
|---|---|
| Gelatin | 35.0 |
| Glycerin | 17.5 |
| Water | 20.0 |
| Hydroxypropyl methyl cellulose phthalate | 7.5 |
| Ammonia solution (25% w/v) | 20.0 |

Gelatin mass is prepared by dispersing gelatin in a mixture of water and glycerin maintained at 70° C. Hydroxypropyl methylcellulose phthalate is dissolved by stirring in to ammonia solution at room temperature. The polymer solution is added to gelatin mass while stirring the mass maintained at 45-50° C. Vacuum is applied to the mixing vessel to remove the ammonia evolved and to obtain bubble free transparent mixture of polymer solution and gelatin mass.

b) Composition of the Medicament:

| Name of the ingredient | mg/Capsule |
|---|---|
| Soybean oil | 280.0 |
| Esomeprazole | 20.0 |
| Meglumine | 20.0 |
| Lecithin | 30.0 |

Lecithin is dispersed into soybean oil using a mechanical stirrer. Esomeprazole and meglumine are added to the dispersion while stirring to obtain a smooth dispersion.
   Manufacturing of Capsule;
This gelatin mixture is transferred to the holding tank of a rotary die capsulation machine for manufacture of a capsule shell. The dispersion-containing medicament is transferred to the hopper of the capsulation machine for filling into the soft gel capsules. The soft gel capsules are manufactured by a rotary die process.

### EXAMPLE—2

a) Composition of the Soft Gelatin Shell:

| Name of the ingredient | Percent by wt. |
|---|---|
| Gelatin | 30.0 |
| Glycerin | 15.0 |
| Water | 20.0 |
| Hydroxypropyl methyl cellulose phthalate | 10.0 |
| Ammonia solution (25% w/v) | 25.0 |

Gelatin mass is prepared by dispersing gelatin in a mixture of water and glycerin maintained at 70° C. Hydroxypropyl methylcellulose phthalate is dissolved by stirring in to ammonia solution at room temperature. The polymer solution is added to gelatin mass while stirring the mass maintained at 45-50° C. Vacuum is applied to the mixing vessel to remove the ammonia evolved and to obtain bubble free transparent mixture of polymer solution and gelatin mass.

b) Composition of the Medicament:

| Name of the ingredient | mg/Capsule |
|---|---|
| Soybean oil | 300.0 mg |
| Esomeprazole | 10.0 mg |
| Meglumine | 10.0 mg |
| Lecithin | 30.0 mg |

Lecithin is dispersed into soybean oil using a mechanical stirrer. Esomeprazole and meglumine are added to the dispersion while stirring to obtain a smooth dispersion.
   Manufacturing of Capsule:
This gelatin mixture is transferred to the holding tank of a rotary die capsulation machine for manufacture of a capsule shell. The dispersion-containing medicament is transferred to the hopper of the capsulation machine for filling into the soft gel capsules. The soft gel capsules are manufactured by a rotary die process.

### EXAMPLE—3

Composition of the Soft Gelatin Shell:

| Name of the ingredient | Percent by wt. |
|---|---|
| Gelatin | 30.0 |
| Glycerin | 15.0 |
| Water | 20.0 |
| Hydroxypropyl methyl cellulose phthalate | 10.0 |
| Ammonia solution (25% w/v) | 25.0 |

Gelatin mass is prepared by dispersing gelatin in a mixture of water and glycerin maintained at 70° C. Hydroxypropyl methylcellulose phthalate is dissolved by stirring in to ammonia solution at room temperature. The polymer solution is added to gelatin mass while stirring the mass maintained at 45-50° C. Vacuum is applied to the mixing vessel to remove the ammonia evolved and to obtain bubble free transparent mixture of polymer solution and gelatin mass.

Composition of the Medicament:

| Name of the ingredient | mg/Capsule |
|---|---|
| Soybean oil | 300.0 mg |
| Rabeprazole sodium | 10.0 mg |
| Meglumine | 10.0 mg |
| Lecithin | 30.0 mg |

Lecithin is dispersed into soybean oil using a mechanical stirrer. Rabeprazole sodium and meglumine are added to the dispersion while stirring to obtain a smooth dispersion.
   Manufacturing of Capsule:
This gelatin mixture is transferred to the holding tank of a rotary die capsulation machine for manufacture of a capsule shell. The dispersion-containing medicament is transferred to the hopper of the capsulation machine for filling into the soft gel capsules. The soft gel capsules are manufactured by a rotary die process.

### EXAMPLE—4

a) Composition of the Soft Gelatin Shell:

| Name of the ingredient | Percent by wt. |
|---|---|
| Gelatin | 40.0 |
| Glycerin | 17.5 |
| Water | 20.0 |
| Hydroxypropyl methyl cellulose phthalate | 5.0 |
| Ammonia solution (25% w/v) | 17.5 |

Gelatin mass is prepared by dispersing gelatin in a mixture of water and glycerin maintained at 70° C. Hydroxypropyl methyl cellulose phthalate is dissolved by stirring in to ammonia solution at room temperature. The polymer solution is added to gelatin mass while stirring the mass maintained at 45-50° C. Vacuum is applied to the mixing vessel to remove the ammonia evolved and to obtain bubble free transparent mixture of polymer solution and gelatin mass.

b) Composition of the Medicament:

| Name of the ingredient | mg/Capsule |
|---|---|
| Soybean oil | 280.0 mg |
| Esomeprazole | 20.0 mg |
| Meglumine | 20.0 mg |
| Gelucire 33/01 | 30.0 mg |

Gelucire 33/01 (glycerol esters of saturated C8-C18 fatty acids) is dispersed into soybean oil using a mechanical stirrer. Esomeprazole and meglumine are added to the dispersion while stirring to obtain a smooth dispersion.
   Manufacturing of Capsule:
This gelatin mixture is transferred to the holding tank of a rotary die capsulation machine for manufacture of a capsule shell. The dispersion-containing medicament is transferred to the hopper of the capsulation machine for filling into the soft gel capsules. The soft gel capsules are manufactured by a rotary die process.

### EXAMPLE—5

a) Composition of the Soft Gelatin Shell:

| Name of the ingredient | Percent by wt. |
|---|---|
| Gelatin | 35.0 |
| Glycerin | 17.5 |
| Water | 25.0 |
| Hydroxypropyl methyl cellulose phthalate | 7.5 |
| Ammonia solution (25% w/v) | 15.0 |

Gelatin mass containing hydroxypropyl methyl cellulose phthalate is prepared by dispersing hydroxypropyl methyl cellulose phthalate in the form of a fine powder in a mixture of glycerin and water maintained at 70° C. in which gelatin is dispersed to dissolve forming the gelatin mass. After cooling the mass to 45° C., ammonia solution is added slowly along the stirrer rod while stirring into the gelatin preparation tank. Stirring is continued till hydroxypropyl methyl cellulose phthalate is completely dissolved. The mass is made bubble free by applying vacuum while maintaining the mass at 45-50° C. under continuous mixing.

b) Composition of the Medicament:

| Name of the ingredient | mg/capsule |
|---|---|
| Soybean oil | 210.0 mg |
| Rabeprazole sodium | 20.0 mg |
| Cremophor RH 40 | 40.0 mg |
| Disodium hydrogen orthophosphate | 30.0 mg |
| Anhydrous | |

Cremophor RH 40 is dispersed in soybean oil at 30° C. After cooling to room temperature rabeprazole sodium and disodium hydrogen orthophosphate are dispersed in to the mixture in the form of fine particles with the help of a mechanical stirrer and/or a homogeniser.
   Manufacturing of Capsule:
This gelatin mixture is transferred to the holding tank of a rotary die capsulation machine for manufacture of a capsule shell. The dispersion-containing medicament is transferred to the hopper of the capsulation machine for filling into the soft gel capsules. The soft gel capsules are manufactured by a rotary die process.

### EXAMPLE—6

a) Composition of the Soft Gelatin Shell:

| Name of the ingredient | Percent by wt. |
|---|---|
| Gelatin | 35.0 |
| Glycerin | 15.0 |
| Water | 20.0 |
| Hydroxypropyl methyl cellulose phthalate | 10.0 |
| Sodium hydroxide solution 1% w/v | 20.0 |

Gelatin mass is prepared by dispersing gelatin in a mixture of water and glycerin maintained at 70° C. Hydroxypropyl methylcellulose phthalate is dissolved by stirring in to sodium hydroxide solution at room temperature. Hydroxypropyl methylcellulose phthalate solution is added to gelatin mass while stirring the mass maintained at 45-50° C. Vacuum is applied to the mixing vessel to remove the ammonia evolved and to obtain bubble free transparent mixture of polymer solution and gelatin mass.

B) Composition of the Medicament:

| Name of the ingredient | mg/capsule |
|---|---|
| Soybean oil | 180.0 mg |
| Rabeprazole sodium | 40.0 mg |
| Hydrogenated vegetable oil | 85.0 mg |
| Gelucire 33/01 | 20.0 mg |
| Meglumine | 40.0 mg |

Hydrogenated vegetable oil is melted and dispersed into soybean oil at 30-40° C. followed by Gelucire 33/01 (glycerol esters of saturated C8-C18 fatty acids), meglumine and rabeprazole sodium and cooled to room temperature. The mixture is kneaded into a smooth paste using a triple roller mill.
   Manufacturing of Capsule:
This gelatin mixture is transferred to the holding tank of a rotary die capsulation machine, for manufacture of a capsule shell. The dispersion-containing medicament is transferred to the hopper of the capsulation machine for filling into the soft gel capsules. The soft gel capsules are manufactured by a rotary die process.

### EXAMPLE—7

a) Composition of the Soft gelatin Shell:

| Name of the ingredient | Percent by wt. |
|---|---|
| Gelatin | 30.0 |
| Propylene glycol | 15.0 |
| Water | 20.0 |
| Hydroxypropyl methyl cellulose phthalate | 10.0 |

Gelatin mass is prepared by dispersing in water at 70° C. Hydroxypropyl methylcellulose phthalate is dissolved in propylene glycol at 60-70° C. and mixed with the gelatin mass to obtain uniform mixture.

b) Composition of the Medicament:

| Name of the ingredient | mg/Capsule |
|---|---|
| Soybean oil | 280.0 mg |
| Esomeprazole | 20.0 mg |
| Meglumine | 20.0 mg |
| Lecithin | 30.0 mg |

Lecithin is dispersed into soybean oil using a mechanical stirrer. Esomeprazole and meglumine are added to the dispersion while stirring to obtain a smooth dispersion.
   Manufacturing of Capsule:
This gelatin mixture is transferred to the holding tank of a rotary die capsulation machine for manufacture of a capsule shell. The dispersion-containing medicament is transferred to the hopper of the capsulation machine for filling into the soft gel capsules. The soft gel capsules are manufactured by a rotary die process.

### EXAMPLE—8

a) Composition of the Soft Gelatin Shell:

| Name of the ingredient | Percent by wt. |
|---|---|
| Gelatin | 35.0 |
| Glycerin | 17.5 |
| Water | 20.0 |
| Polyvinylacetate phthalate (PVAP) | 7.5 |
| Ammonia solution (25% w/v) | 20.0 |

Gelatin mass is prepared by dispersing gelatin in a mixture of water and glycerin maintained at 70° C. Polyvinylacetate phthalate is dissolved by stirring into ammonia solution at room temperature. Polyvinylacetate phthalate solution in ammonia is added to gelatin mass while stirring the mass maintained at 45-50° C. Vacuum is applied to the mixing vessel to remove the ammonia evolved and to obtain bubble free transparent mixture of polymer solution and gelatin mass.

B) Composition of the Medicament:

| Name of the ingredient | mg/capsule |
|---|---|
| Sunflower oil | 200.0 mg |
| Esomeprazole | 30.0 mg |
| Cremophor RH 40 | 40.0 mg |
| Disodium hydrogen orthophosphate | 30.0 mg |
| Anhydrous | |

Cremophor RH 40 is dispersed in sunflower oil at 30° C. After cooling to room temperature esomeprazole and disodium hydrogen orthophosphate are dispersed into the mixture in the form of fine particles with the help of a mechanical stirrer and/or a homogeniser.
   Manufacturing of Capsule:
This gelatin mixture is transferred to the holding tank of a rotary die capsulation machine for manufacture of a capsule shell. The dispersion-containing medicament is transferred to the hopper of the capsulation machine for filling into the soft gel capsules. The soft gel capsules are manufactured by a rotary die process.

### EXAMPLE—9

Composition of the Soft Gelatin Shell:

| Name of the ingredient | Percent by wt. |
|---|---|
| Gelatin | 35.0 |
| Glycerin | 17.5 |
| Water | 20.0 |
| Polyvinylacetate phthalate (PVAP) | 7.5 |
| Ammonia solution (25% w/v) | 20.0 |

Gelatin mass is prepared by dispersing gelatin in a mixture of water and glycerin maintained at 70° C. Polyvinylacetate phthalate is dissolved by stirring into ammonia solution at room temperature. Polyvinylacetate phthalate solution in ammonia is added to gelatin mass while stirring the mass maintained at 45-50° C. Vacuum is applied to the mixing vessel to remove the ammonia evolved and to obtain bubble free transparent mixture of polymer solution and gelatin mass.

b) Composition of the Medicament:

| Name of the ingredient | mg/capsule |
|---|---|
| Sunflower Oil | 185.7 |
| Esomeprazole | 20.0 |
| Meglumine | 20.0 |
| Gelucire 33/01 | 13.00 |
| Docusate Sodium | 20.00 |
| Colloidal silicon-dioxide | 1.30 |
| Microcrystalline Cellulose | 10.00 |

Meglumine, esomeprazole along with colloidal silicon-dioxide are dispersed in sunflower oil, microcrystalline cellulose, Gelucire 33/01 and docusate sodium are added to this mixture and stirred at low speed to ensure a uniform suspension.

Manufacturing of Capsule:

The gelatin mixture is transferred to the holding tank of a rotary die capsulation machine for manufacture of a capsule shell. The dispersion-containing medicament is transferred to the hopper of the capsulation machine for filling into the soft gel capsules. The soft gel capsules are manufactured by a rotary die process.

The advantages of the present invention are:

1) Simple method of manufacturing, when compared to the methods disclosed in the prior art making the process economical.
2) Improved bioavailability when compared to the solid enteric coated pellets and tablets as the medicament is solublised or suspended in the form of very fine particles in the liquid/semisolid pharmaceutical composition filled into the soft gel capsule.
3) The reactive acidic groups of enteric polymers are in minimal contact with the active ingredient as the polymer is mixed into large amount of gelatin mass. Only small amounts of alkaline reactive material is required to neutralize the free fatty acids in the oily substances and free acidic reacting groups of enteric polymer in contact with the active ingredient on inner surface of the shell.
4) The soft gel does not require any protective sub-coating. Consequently the active ingredient quickly dissolves into the intestinal fluid once the gastric resistant but intestinal soluble gelatin composition is dissolved.
5) The soft gel capsules are simple in composition and therefore do not require any sophisticated equipment for manufacturing.

1) A pharmaceutical composition in form of a soft gel capsule resistant to gastric juice and soluble in intestine useful for the treatment of duodenal ulcers and related ailments which comprises of a gelatin shell resistant to gastric juice and soluble in intestine having an enteric polymer mixed into gelatin in the form of free acid or its salt and the capsule incorporating a composition comprising enantiomers of omeprazole such as esomeprazole, rabeprazole or its salts or its derivatives or their mixtures, a hydrophobic oily substance or a mixture of such oily substances, an alkaline inert reacting material, a suspending agent, a surface active agent and/or a solublising agent; wherein the resulting capsules being insoluble in aqueous medium up to a pH of 5.5 but quickly dissolving above pH of 6.0.

2) A pharmaceutical composition as claimed in claim 1 wherein the amount of enetiomers of omeprazole or its salts or derivatives or their mixtures present in the formulation is equivalent to one unit dose.

3) A pharmaceutical composition as claimed in claim 1 wherein the enteric polymer employed for coating the gelatin shell is selected from polymers such as hydroxypropyl methyl cellulose phthalate, alkyl methacrylate and methacrylic acid copolymers, polyvinyl acetate phthalate and the like.

4) A pharmaceutical composition as claimed in claim 3 wherein the enteric polymer employed is in the form of free acid or their ammonia or alkali metal salts.

5) A pharmaceutical composition as claimed in claim 3 wherein the amount employed ranging from 2.0 to 40.0 percent, preferably 5.0 to 25.0 percent by weight, with reference to the dried shell.

6) A pharmaceutical composition as claimed in claim 1 wherein the enantiomers of omeprazole or its salts or derivatives or their mixtures employed in the formulation is suspended/solubilised in a hydrophobic oily substance selected from fats and oils of vegetable origin such as sesame oil, corn oil, maize oil, soybean oil, sunflower oil, arachis oil, gingly oil and the like; animal origin such as fish oil, pig oil, beef oil and the like; esters of straight chain aliphatic oils such as Sunsoft 700 P-2 (Taiho chemical company) Panasete 810 (Nippon oils and Fats); hydrogenated vegetable oils or a mixture thereof.

7) A pharmaceutical composition as claimed in claim 6 wherein the amount of hydrophobic oily substance used ranging from 25.0 to 95.0 percent, preferably 35.0 to 90 percent by weight with reference to the contents filled in capsules.

8) A pharmaceutical composition as claimed in claim 1 wherein substances such as colloidal silicon dioxide, polyvinylpyrrolidone, microcrystalline cellulose are used as dispersing agents in an amount ranging from 0.5 to 20.0 percent preferably 1.0 to 10.0 percent by weight and materials such as lecithin, polyoxyethylene castor oil derivative such as Cremophor RH 40 (polyoxyl 40 hydrogenated castor oil), Cremophor EL (polyoxyl 35 castor oil, BASF) polyoxyethylene sorbitan fatty acid esters, Gelucire 33/01 (glycerol esters of fatty acids), sodium lauryl sulphate, docusate sodium and the like are used as surface active agent and/or a solublising agent.

9) A pharmaceutical composition as claimed in claim 8 wherein the amount of surface active agent and/or solublising agent ranging from 2.0 to 20.0 percent, preferably 4.0 to 15.0 percent by weight, with reference to the contents filled in capsule.

10) A pharmaceutical composition as claimed in claim 1 wherein materials such as the sodium, potassium, calcium, magnesium and aluminium salts of phosphoric acid, carbonic acid, citric acid, other suitable organic or inorganic acids; meglumine, triethanolamine and the like are used as alkaline inert reacting materials.

11) A pharmaceutical composition as claimed in claim 1 wherein the amount ranging from 5.0 to 40.0 percent, preferably 10.0 to 25.0 percent by weight, with reference to the contents filled in capsule.

12) A pharmaceutical composition as claimed in claim 1 wherein the soft gel capsules are treated with a gelatin cross linking agent such as formaldehyde, glutaraldehyde, crotonaldehyde, 1,2-phthalic acid aldehyde, 1,3-phthalic acid aldehyde, 1,4-phthalic acid aldehyde; carboimides such as 1-ethyl-3-[2-morpholinyl-(4) -ethyl]-carboimide-metho-P-toluene-sulfonate and the like.

13) A pharmaceutical composition as claimed in claim 1 wherein the soft gel capsules are treated with cold dilute solutions of acids selected from hydrochloric acid, sulphuric acid, nitric acid, phosphoric acid, citric acid, propionic acid, benzoic acid, oxalic acid, maleic acid, fumaric acid and the like.

14) A process for the preparation of a pharmaceutical composition in the form of a soft gel capsule resistant to gastric juice and soluble in intestine useful for the treatment of duodenal ulcers and related ailments which comprises forming a gelatin shell which is resistant to gastric juice and soluble in intestine having an enteric polymer in the form of free acid or its salt, and incorporating into the resultant capsule a composition comprising esomeprazole or rabeprazole or other enantiomers of omeprazole or its salts or derivatives or their mixtures, a hydrophobic oily substance or a mixture of such substances, an alkaline inert reacting material, a suspending agent, a surface active agent and/or a solublising agent; wherein the resultant capsules being insoluble in aqueous medium up to a pH of 5.5 but quickly dissolve above pH of 6.0.

15) A process as claimed in claim 14 wherein the amount of esomeprazole or rabeprazole or other enantiomers of omeprazole or its salts or derivatives or their mixtures present in the formulation is equivalent to one unit dose.

16) A process as claimed in claim 14 wherein the enteric polymer employed for coating the gelatin shell is selected from polymers such as hydroxypropyl methyl cellulose phthalate, alkyl methacrylate and methacrylic acid copolymers, polyvinyl acetate phthalate and the like.

17) A process as claimed in claim 14 wherein the enteric polymer employed is in the form of free acid or their ammonia or alkali metal salts.

18) A process as claimed in claim 16 wherein the amount employed ranging from 2.0 to 40.0 percent, preferably 5.0 to 25.0 percent by weight, with reference to the dried shell.

19) A pharmaceutical composition as claimed in claim 14 wherein the enantiomers of omeprazole or its salts or derivatives or their mixtures employed in the formulation is suspended/solubilised in a hydrophobic oily substance selected from fats and oils of vegetable origin such as sesame oil, corn oil, maize oil, soybean oil, sunflower oil, arachis oil, gingly oil and the like; animal origin such as fish oil, pig oil, beef oil and the like; esters of straight chain aliphatic oils such as Sunsoft 700 P-2 (Taiho chemical company) Panasete 810 (Nippon oils and Fats); hydrogenated vegetable oils or a mixture thereof.

20) A process as claimed in claim 19 wherein the amount of hydrophobic oily substance used ranging from 25.0 to 95.0 percent, preferably 35.0 to 90.0 percent by weight, with reference to the contents filled in capsules.

21) A process as claimed in claim 14 wherein substances such as colloidal silicon dioxide, polyvinylpyrrolidone, microcrystalline cellulose are used as dispersing agents in an amount ranging from 0.5 to 20.0 percent preferably 1.0 to 10.0 percent by weight and materials such as lecithin, polyoxyethylene castor oil derivative such as Cremophor RH 40 (polyoxyl 40 hydrogenated castor oil), Cremophor EL (polyoxyl 35 castor oil, BASF), Gelucire 33/01 (glycerol esters of fatty acids), polyoxyethylene sorbitan fatty acid esters, sodium lauryl sulphate, docusate sodium and the like are used as surface active agent and/or a solublising agent.

22) A process as claimed in claim 21 wherein the amount of surface active agent and/or solublising agent ranging from 2.0 to 20.0 percent, preferably 4.0 to 15.0 percent by weight, with reference to the contents filled in capsule.

23) A pharmaceutical composition as claimed in claim 14 wherein materials such as the sodium, potassium, calcium, magnesium and aluminium salts of phosphoric acid, carbonic acid, citric acid, other suitable organic or inorganic acids; meglumine, triethanolamine and the like are used as alkaline inert reacting materials.

24) A process as claimed in claim 14 wherein the amount ranging from 5.0 to 40.0 percent, preferably 10.0 to 25.0 percent by weight, with reference to the contents filled in capsule.

25) A process as claimed in claim 14 wherein the soft gel capsules are treated with a gelatin cross linking agent such as formaldehyde, glutaraldehyde, crotonaldehyde, 1,2-phthalic acid aldehyde, 1,3-phthalic acid aldehyde, 1,4-phthalic acid aldehyde; carboimides such as 1-ethyl-3-[2-morpholinyl-(4)-ethyl]-carboimide-metho-P-toluene-sulfonate and the like.

26) A process as claimed in claim 14 wherein the soft gel capsules are treated with cold dilute solutions of acids selected from hydrochloric acid, sulphuric acid, nitric acid, phosphoric acid, citric acid, propionic acid, benzoic acid, oxalic acid, maleic acid, fumaric acid and the like.
